(19) United States
(12) Patent Application Publication
(10) Pub. No.: US 2017/0239091 A1
(43) Pub. Date: Aug. 24, 2017

(54) EARDRUM INCISION DEVICE

(71) Applicant: ALTEK CORPORATION, Hsinchu City (TW)

(72) Inventors: Douglas Franz, Sioux falls, SD (US); Chin-Cheng Chiu, Hsinchu City (TW); Jui-Yuan Yu, Miaoli County (TW); Hsi-Hsin Loo, Taipei City (TW)

(73) Assignee: ALTEK CORPORATION, Hsinchu City (TW)

(21) Appl. No.: 15/438,772

(22) Filed: Feb. 22, 2017

Related U.S. Application Data
(60) Provisional application No. 62/298,427, filed on Feb. 22, 2016.
(60) Provisional application No. 62/298,432, filed on Feb. 22, 2016.

(30) Foreign Application Priority Data
Feb. 8, 2017 (TW) 106104031

(51) Int. Cl. A61F 11/00; A61B 17/3209; A61B 1/227; A61M 1/00; A61B 1/06; A61B 1/045; A61B 1/00; A61B 17/32; A61B 1/04

(52) U.S. Cl. CPC A61F 11/004; A61B 17/320016; A61B 17/3209; A61B 1/227; A61B 1/042; A61B 1/0684; A61B 1/0661; A61B 1/045; A61B 1/00188; A61B 1/00094; A61B 1/00124; A61B 1/00147; A61M 1/0086

(57) ABSTRACT

An eardrum incision device includes a main body, a replaceable inserting portion and a display device. The replaceable inserting portion is detachably connected to the main body. The replaceable inserting portion has an insertion end located on a side far away from the main body and adapted to be inserted into an ear canal of a user. The replaceable inserting portion includes an image-capturing device, a mounting hole and a replaceable incision component. The image-capturing device is disposed at the insertion end. The mounting hole extends in a direction from the insertion end toward the main body. The replaceable incision component is removably disposed in the mounting hole and adapted to pierce out of the mounting hole in a direction toward the insertion end, so as to incise the eardrum of the user. The display device is disposed on the main body and electrically connected to the image-capturing device.

### CROSS-REFERENCE TO RELATED APPLICATION

This application claims the priority benefits of U.S. provisional application Ser. No. 62/298,432, filed on Feb. 22, 2016, U.S. provisional application Ser. No. 62/298,427, filed on Feb. 22, 2016 and Taiwan application serial no. 106104031, filed on Feb. 8, 2017. The entirety of each of the above-mentioned patent applications is hereby incorporated by reference herein and made a part of this specification.

### BACKGROUND OF THE INVENTION

1. Field of the Invention

The invention relates to an eardrum incision device and particularly relates to an eardrum incision device having an image-capturing component and a display.

2. Description of Related Art

Eardrum incision surgery is an effective and easy treatment for otitis media. Among the existing technologies, an optical image-capturing component is usually employed to observe the inside of an ear canal. Alternatively, other image-capturing components are employed to probe into the ear canal.

However, among the current medical instruments for eardrum incision surgery, the image-capturing component, an image display device and an incision component are usually limited to the design of an instrument or the volume of a device and therefore may not be compatible with each other in the same instrument. Consequently, it usually requires different instruments and steps that are taken in sequence to observe the inside of the ear canal and conduct eardrum incision, and it is not possible to conduct eardrum incision and observe the inside of the ear canal at the same time. Besides, the main bodies of the incision component and the instrument are connected to or integrated with each other. Therefore, whenever the eardrum incision surgery is conducted, the incision component should be washed and sterilized immediately to prevent the transmission and infection of viruses. However, washing and sterilizing the incision component increases labor and time required by the eardrum incision surgery.

### SUMMARY OF THE INVENTION

The invention provides an eardrum incision device having an image-capturing component and a display that are employed to observe the inside of an ear canal while an eardrum is incised.

The eardrum incision device of the invention includes a main body, a replaceable inserting portion and a display. The replaceable inserting portion is detachably connected to the main body. The replaceable inserting portion has an insertion end located at an end far away from the main body and adapted to be inserted into an ear canal of a user. The replaceable inserting portion includes an image-capturing component, a mounting hole and a replaceable incision component. The image-capturing component is disposed at the insertion end. The mounting hole extends in a direction from the insertion end toward the main body. The replaceable incision component is removably disposed in the mounting hole and adapted to pierce out of the mounting hole in a direction toward the insertion end, so as to incise the eardrum of the user. The display is disposed on the main body and electrically connected to the image-capturing component to display an image captured by the image-capturing component.

According to an embodiment of the invention, the image-capturing component includes a dual-lens camera module.

According to an embodiment of the invention, the eardrum incision device further includes a fixing ring that is disposed between the replaceable inserting portion and the main body. The replaceable inserting portion is detachably fixed on the main body through the fixing ring.

According to an embodiment of the invention, the eardrum incision device further includes a fixing part. The fixing part is detachably put on the replaceable inserting portion and includes an ear-opening element and a holding element. The ear-opening element is detachably inserted into the ear canal. The holding device is disposed at an end of the ear-opening element that is inserted into the ear canal, and movably leans against the ear canal. The holding device has a through hole, and the replaceable inserting portion is disposed through the through hole to be fixed in the ear canal.

According to an embodiment of the invention, the eardrum incision device further includes a light source. The light source is disposed at the insertion end. When the insertion end is inserted into the ear canal of the user, the light source is adapted to provide illumination.

According to an embodiment of the invention, the eardrum incision device further includes a driving element. The driving element is disposed in the mounting hole and coupled to the replaceable incision component. The driving element is adapted to drive the replaceable incision component to pierce out of the mounting hole toward the insertion end.

According to an embodiment of the invention, the eardrum incision device further includes a suction element. The suction element is disposed in the replaceable incision component.

According to an embodiment of the invention, the eardrum incision device further includes an ear tube. The ear tube is disposed at an end of the suction element.

According to an embodiment of the invention, the eardrum incision device further includes a gripping portion and a switch button. The gripping portion is connected to the main body, and the switch button is disposed on the gripping portion.

According to an embodiment of the invention, the replaceable inserting portion is made of a disposable material.

In view of the foregoing, in the embodiments of the invention, the eardrum incision device has the image-capturing component and the display. Consequently, when the eardrum is incised by using the eardrum incision device, an operator may depend on images captured by the image-capturing component and displayed by the display to observe the inside of the ear canal. In the embodiments of the invention, the replaceable inserting portion of the eardrum incision device and the interior members thereof are designed to be replaceable. Therefore, whenever use of the eardrum incision device is finished, a new replaceable inserting portion may immediately substitute for the old replaceable inserting portion of the eardrum incision device to enhance the hygiene and convenience of using the eardrum incision device.

In order to make the aforementioned and other features and advantages of the invention comprehensible, several exemplary embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1A is an external view of an eardrum incision device according to an embodiment of the invention.

FIG. 1B is a side view of a display of the eardrum incision device of FIG. 1A.

FIG. 1C is a schematic view of an insertion end of a replaceable inserting portion of the eardrum incision device of FIG. 1A.

FIGS. 2A and 2B are an embodiment of the replaceable inserting portion of the eardrum incision device of FIG. 1A.

FIG. 3 is a schematic view of a partial member of the eardrum incision device of FIG. 1A.

FIGS. 4A and 4B are cross-sectional views of the partial member of the eardrum incision device of FIG. 1A.

FIG. 5A is an exploded view of a fixing part of the eardrum incision device of FIG. 1A.

FIGS. 5B and 5C are schematic views of an embodiment of the fixing part of the eardrum incision device of FIG. 1A.

FIG. 6 is a schematic view of an embodiment of the eardrum incision device of FIG. 1A.

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

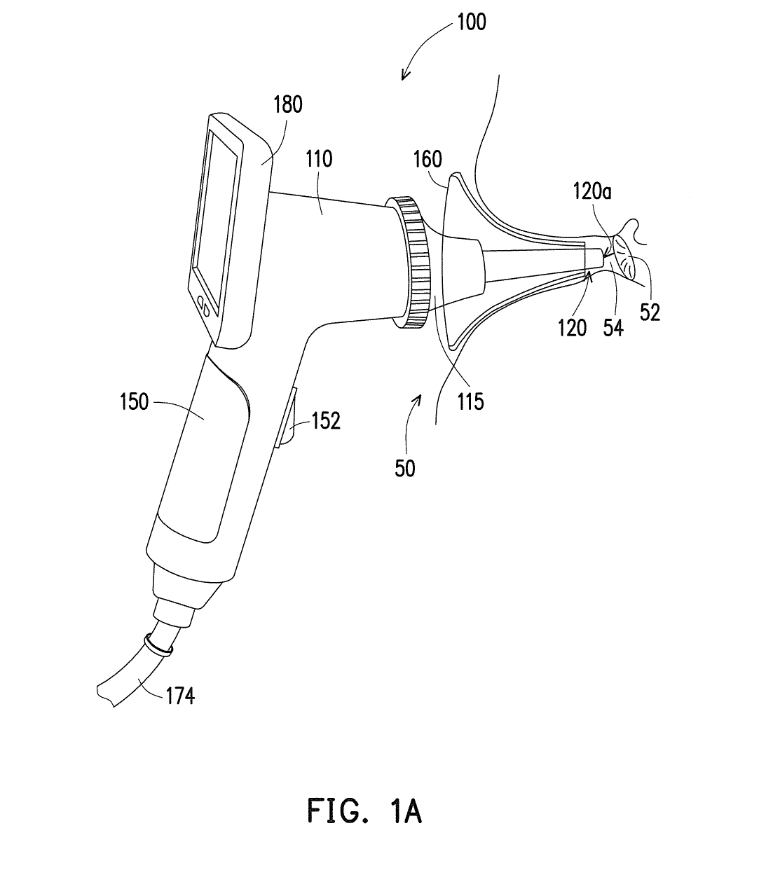

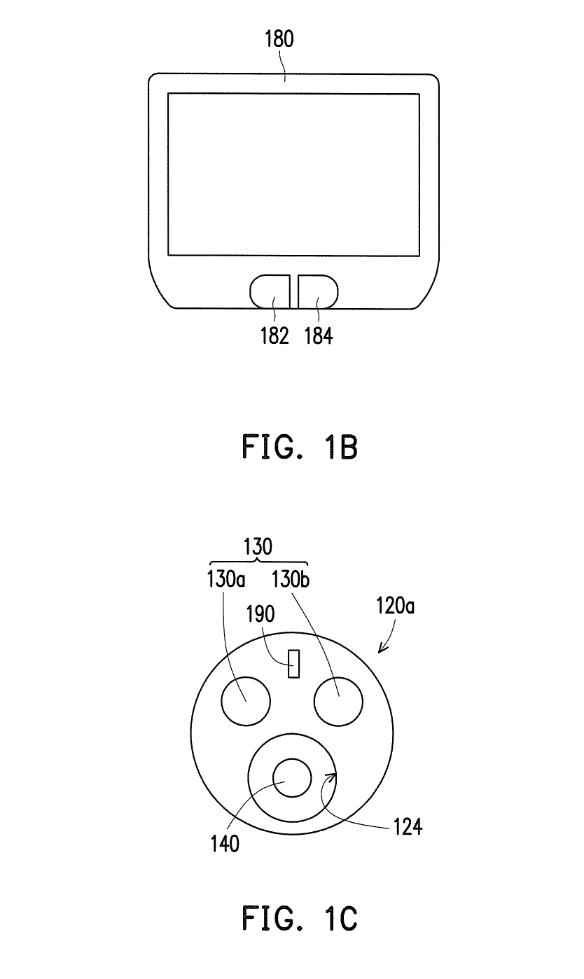

FIG. 1A is an external view of an eardrum incision device according to an embodiment of the invention. FIG. 1B is a side view of a display of the eardrum incision device of FIG. 1A. FIG. 1C is a schematic view of an insertion end of a replaceable inserting portion of the eardrum incision device of FIG. 1A. FIGS. 1A and 1B and 1C are provided for reference. In the embodiment, an eardrum incision device 100 includes a main body 110, a replaceable inserting portion 120 and a display 180. The replaceable inserting portion 120 is detachably connected to the main body 110 and has an insertion end 120a. Besides, the insertion end 120a is located at an end far away from the main body 110 and adapted to be inserted into an ear canal 54 of a user (not shown).

The replaceable inserting portion 120 includes an image-capturing component 130, a mounting hole 124 and a replaceable incision component 140. The image-capturing component 130 is disposed at the insertion end 120a of the replaceable inserting portion 120. In the embodiment, the image-capturing component 130 may have a dual-lens camera module, such as a left lens 130a and a right lens 130b as shown in FIG. 1C. Besides, the mounting hole 124 from the inserting portion extends in a direction from the insertion end toward the main body. In the embodiment, the image-capturing component 130 includes dual lenses (the left lens 130a and the right lens 130b) at the same time. Therefore, the eardrum incision device 100 may capture a three-dimensional image in the ear canal 54 through the dual lenses of the image-capturing component 130. A distance between the insertion end 120a of the replaceable inserting portion 120 and an eardrum 52, i.e. a depth at which the insertion end 120 is inserted into the ear canal 54, may be obtained according to the three-dimensional image.

Referring to FIG. 1C again, a replaceable incision component 140 is removably disposed in the mounting hole 124 and may pierce out of the replaceable inserting portion 120 in a direction toward the insertion end 120a to incise the eardrum 52 of the user.

In the embodiment, the left lens 130a and the right lens 130b of the image-capturing component 130 have a diameter size of, for example, less than or equal to 4 mm. Therefore, the left lens 130a and the right lens 130b of the image-capturing component 130 of the embodiment may be disposed at the insertion end 120a of the replaceable inserting portion 120 at the same time. Therefore, in the embodiment, the insertion end 120a of the image-capturing component 130 having a dual-lens camera module has a diameter size of approximately less than or equal to 10 mm and may be inserted into the ear canal 54 of the user.

In the embodiment, the eardrum incision device 100 captures a three-dimensional image in the ear canal 54 through the left lens 130a and the right lens 130b at the same time. Afterwards, the obtained three-dimensional image is analyzed to obtain information on the depth at which the insertion end 120a is the ear canal 54, i.e. a distance between the insertion end 120a and the ear canal 52.

As shown in FIG. 1A, the eardrum incision device 100 of the embodiment further includes a fixing part 160 that is detachably put on the replaceable inserting portion 120. In the embodiment, the fixing part 160 may be firstly placed in the ear canal 54 of the user and flares the ear canal 54 slightly. Next, the replaceable inserting portion 120 of the eardrum incision device 100 may be inserted into the ear canal 54 along the fixing part 160 and fixed at a target position in the ear canal 54 through the fixing part 160. After the replaceable inserting portion 120 of the eardrum incision device 100 is fixed through the fixing part 160, the replaceable inserting portion 120 may no longer move along a depth direction toward the ear canal 54, i.e. a direction toward the eardrum 52, so as to prevent the replaceable inserting portion 120 from contacting the eardrum 52.

As described above, the replaceable inserting portion 120 is detachably disposed on the main body 110, and the fixing part 160 is detachably put on the replaceable inserting portion 120. In other words, the replaceable inserting portion 120 and the fixing part 160 may be removed from the main body 110 after use of the eardrum incision device 100 is finished. In the embodiment, the replaceable inserting portion 120 and the fixing part 160 may be made of, for example, disposable materials. This way may reduce the time and labor required by washing and sterilizing the replaceable inserting portion 120 and the fixing part 160, prevent the risk of mutual infection of genus and viruses among different users through the replaceable inserting portion 120 or the fixing part 160 and enable the user to employ the eardrum incision device 100 more conveniently and hygienically.

As illustrated in FIG. 1A, the eardrum incision device 100 has a gripping portion 150 and a switch button 152. The gripping portion 152 is connected to the main body 110, and the switch button 152 is disposed on the gripping portion 150. In the embodiment, the user may grip the eardrum incision device 100 by the gripping portion 150 and turn on or turn off the eardrum incision device 100 by pressing the switch button 152. Besides, a vacuum line 174 is connected between a vacuum source (not shown) and the gripping portion 150, further extends into the main body 110 and is connected to the replaceable inserting portion 120.

As illustrated in FIG. 1B, in the embodiment, the display 180 is disposed on a side of the main body relative to the replaceable inserting portion 120. The display 180 is electrically connected to the image-capturing component 130 to display the three-dimensional image of the inside of the ear canal 54 filmed by the image-capturing component 130.

In the embodiment, the display 180 may have a first control button 182 and a second control button 184. The first control button 182 is configured to control image capture by the image-capturing component 130, and the second control button 184 is configured to control the replaceable inserting portion 120 to suck waterlogging in the eardrum 52 through the vacuum line 174.

The eardrum incision device 100 further has a light source 190. The light source 190 is also disposed at the insertion end 120a. Therefore, when the insertion end 120a is inserted into the ear canal 54 of the user, the light source 190 provides illumination in the ear canal 54.

In the embodiment, the replaceable incision component 140, after used, may be removed from the mounting hole 124 alone for replacement. Meanwhile, the replaceable incision component 140 is also made of, for example, a disposable material. This way reduces the cost of time and labor required by washing and sterilization, lowers the risk of mutual infection of the germs of the user or the patient through the replaceable incision component 140 and heightens the safety and hygiene of the eardrum incision surgery.

As illustrated in FIG. 1C, the light source 190 is, for example, a light emitting diode (LED) light source or a laser diode light source. The light source 190 is disposed above the dual lenses of the image-capturing component 130 and sticks out of the insertion end 120a to prevent glare caused by the lenses of the image-capturing component 130 capturing images. Furthermore, the light source 190 may be configured to project a fixing mark (not shown) onto the eardrum 52. Afterwards, the image-capturing component 130 of the eardrum incision device 100 may take the fixing mark as a detection target for reference to measure a distance between the insertion end 120a and the eardrum 52. In other words, the fixing mark projected by the light source 190 may lead the user to align himself with a specific position on the eardrum 52 in the image and measure a distance between the insertion end 120a and the specific position.

The illumination of the light source 190 may be projected through a lens having a fixing mark as well. For instance, the fixing mark may be disposed on the bottom of the lens of the image-capturing component 130, and the light source 190 may refract the picture of the fixing mark disposed on the lens of the image-capturing component 130 onto a surface of the eardrum 52 by setting up a pin-hole or slit.

In the embodiment, since the image-capturing component 130 captures images through dual lenses, the filming quality of an image may be effective elevated. In other words, the image-capturing component 130 may boost light sensitivity and reduce noises through the dual lenses.

Referring to FIG. 1A again, in the embodiment, the eardrum incision device 100 may further have a fixing ring 115 that is disposed between the replaceable inserting portion 120 and the main body 110. The replaceable inserting portion 120 is detachably fixed on the main body 110 through the fixing ring 115. Therefore, after use of the eardrum incision device 100 is finished, the replaceable inserting portion 120 may be detached to be washed or replaced. Furthermore, as described above, the replaceable inserting portion 120 and all of the members disposed therein may be made of disposable materials. In this case, the replaceable inserting portion 120 of the eardrum incision device 100 and the interior members thereof may be replaced conveniently, and it is possible to prevent the problem of poor hygiene or infection of viruses for the user due to repeated use of the members.

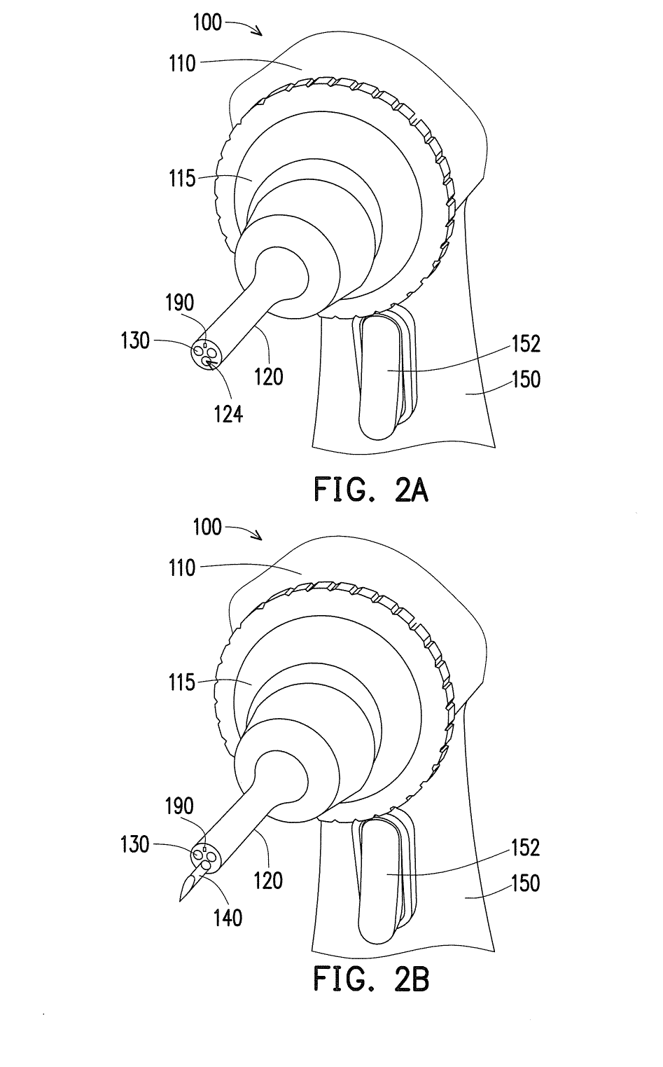

FIGS. 2A and 2B are an embodiment of the replaceable inserting portion of the eardrum incision device of FIG. 1A. FIG. 2A depicts the situation where the replaceable incision component 140 of the eardrum incision device 100 is put in the mounting hole 124, while FIG. 2B depicts the situation where the replaceable incision component 140 of the eardrum incision device 100 pierces out of the mounting hole 124. As illustrated in FIGS. 2A and 2B, a doctor or an operator may press the switch button 152 of the gripping portion 150 to drive the replaceable incision component 140 in the mounting hole 124 and make the replaceable incision component 140 pierce out of the mounting hole 124 in the direction toward the insertion end 120a to incise the eardrum 52 of the user as illustrated in FIG. 1.

Before the eardrum 52 of the user is incised through the replaceable incision component 140, the image-capturing component 130 disposed on the insertion end 120a firstly captures the three-dimensional image of the inside of the ear canal 54 of the user through the dual lenses to obtain information on the depth at which the insertion end 120a is in the ear canal 54. After use of the eardrum incision device 100 is finished, the replaceable incision component 140 may be put back into the mounting hole 124 in the replaceable inserting portion 120 by pressing the switch button 152.

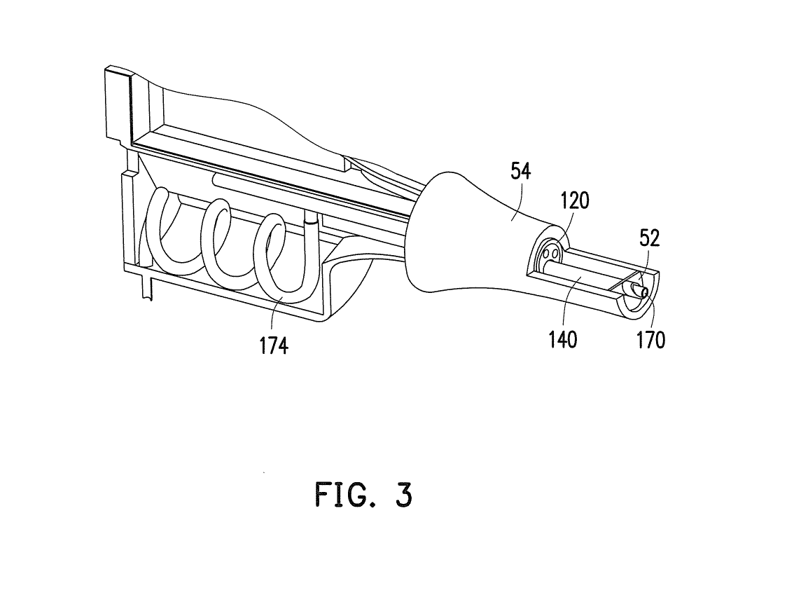

FIG. 3 is a schematic view of a partial member of the eardrum incision device of FIG. 1A. In the embodiment, the eardrum incision device 100 may have a suction element 170 that is disposed in the replaceable incision component 140. That is, the replaceable incision component 140 surrounds the suction element 170. Besides, the suction element 170 may be connected to the vacuum source through the vacuum line 174. Specifically speaking, in the embodiment, after the eardrum incision device 100 incises the eardrum 52 through the replaceable incision component 140, the suction element 170 may move into the eardrum 52 deeply to suck waterlogging out of the eardrum 52 to an ear portion 50 to facilitate the following relevant operations.

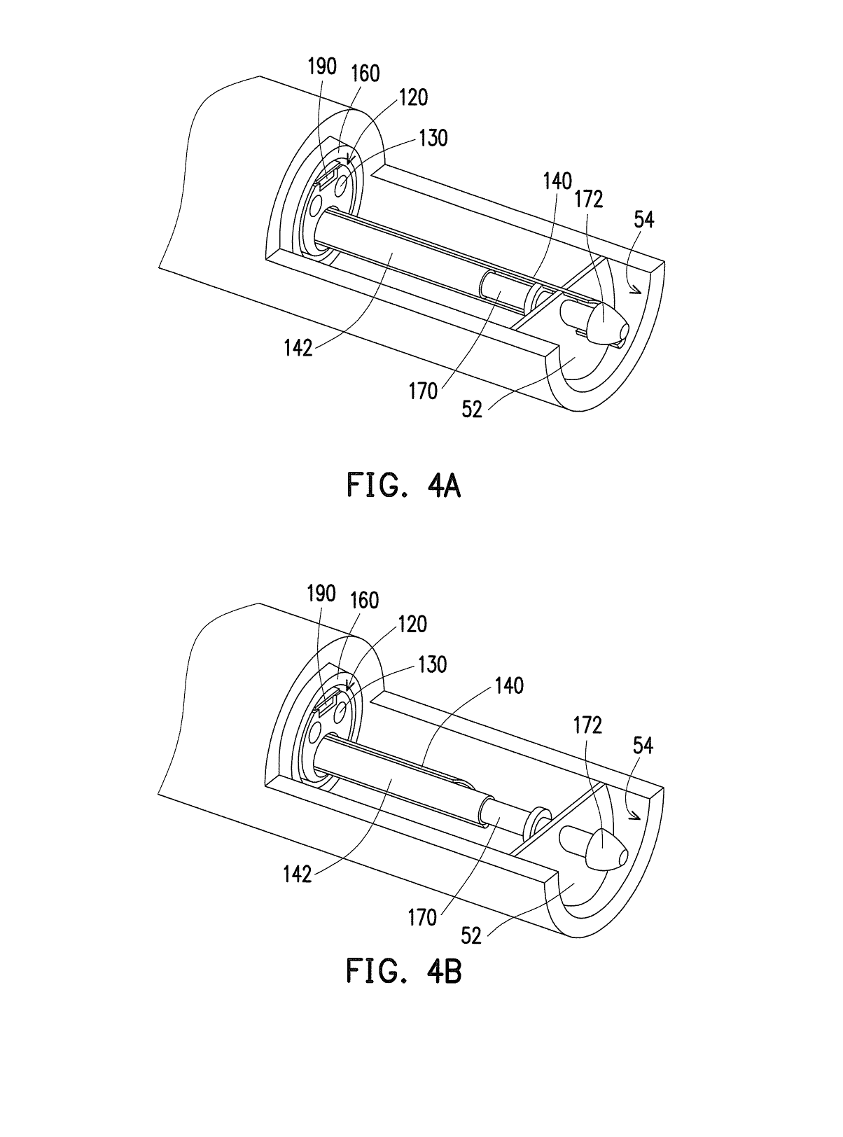

FIGS. 4A and 4B are cross-sectional views of the partial member of the eardrum incision device of FIG. 1A. In the embodiment, an ear tube 172 may be disposed on a side of the suction element 170 disposed in the replaceable incision component 140 near an opening of the replaceable incision component 140. The ear tube 172 leads waterlogging in the eardrum 52 to the suction element 170. As illustrated in FIGS. 4A and 4B, when the replaceable incision component 140 incises the eardrum 52 in the ear canal 54, the replaceable incision component 140 may be driven by a driving element 142 to return in a direction toward the mounting hole 124 in the replaceable inserting portion 120. Meanwhile, the ear tube 172 remains in the eardrum 52 until waterlogging in the eardrum 52 is sucked out by the suction element 170. When waterlogging is sucked out of the eardrum 52, the suction element 170 and the ear tube 172 to which the suction element 170 is connected may return to the mounting hole 124 in the replaceable inserting portion 120 through the driving element 142.

In the embodiment, the replaceable incision component 140 of the eardrum incision device 100 and the suction element 170 may be accommodated together in the mounting hole 124, and the mounting hole 124, the image-capturing component 130 and the light source 190 are disposed together in the replaceable inserting portion 120 of the eardrum incision device 100. Consequently, the volume of the replaceable inserting portion 120 of the eardrum incision device 100 may be effectively reduced and adapted to be inserted into the ear canal 54 of the user. Besides, when the insertion end 120a moves in the ear canal 54, and the replaceable incision component 140 incises the eardrum 52, depths at which the insertion end 120a and the replaceable incision component 140 are in the ear canal 54 may be detected with the aid of the image-capturing component 130.

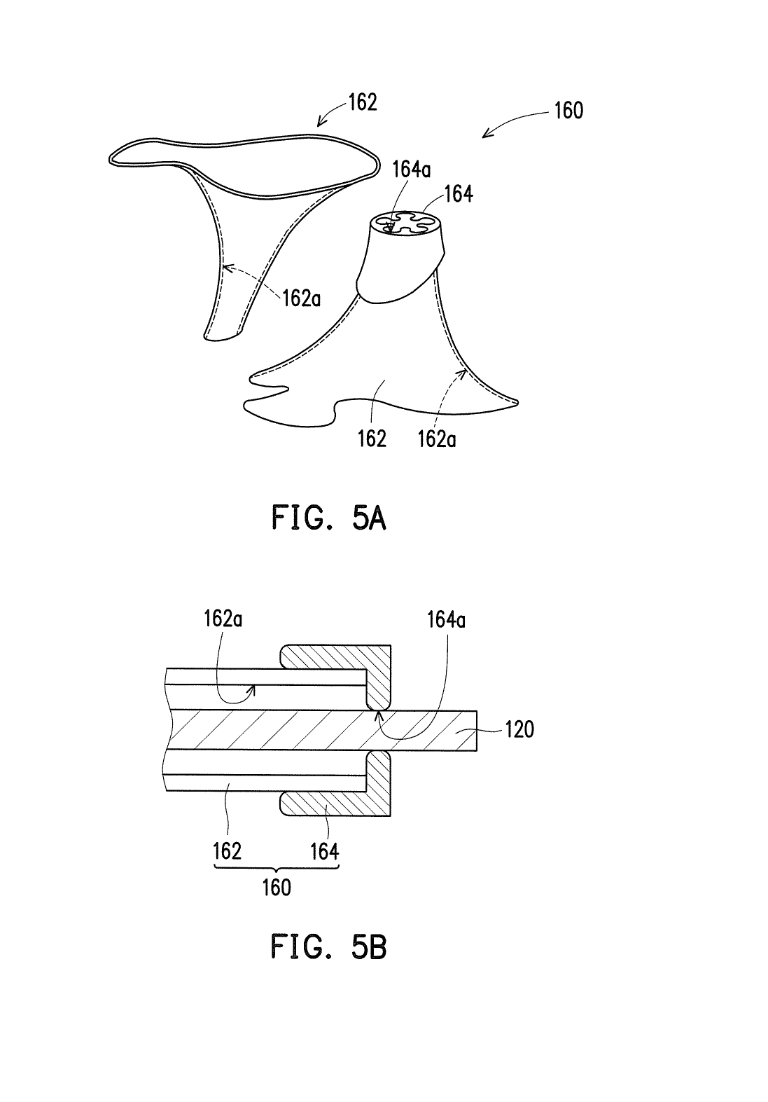

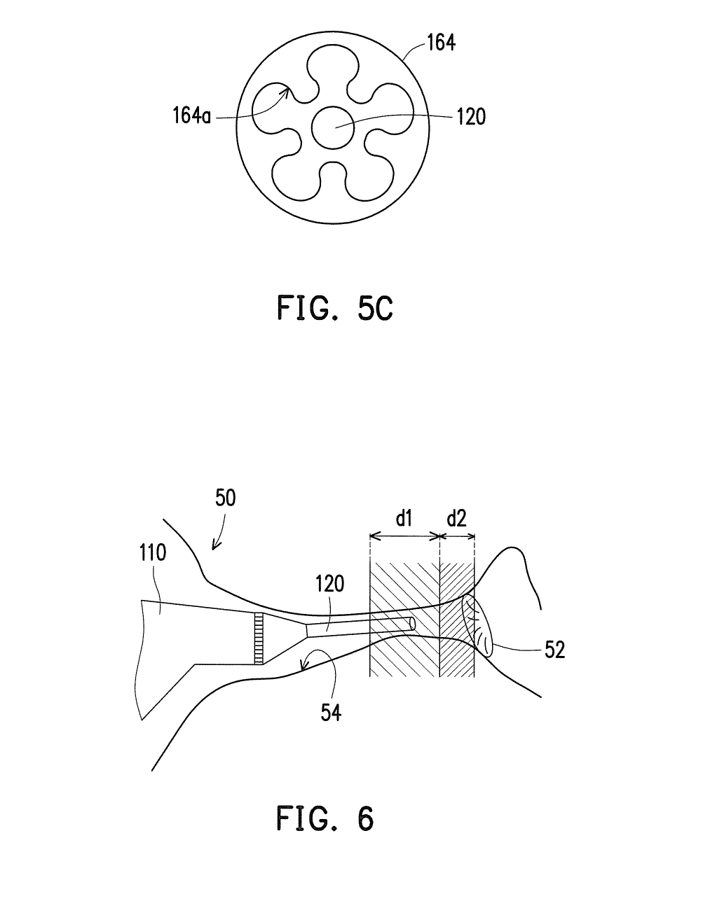

FIG. 5A is an exploded view of the fixing part of the eardrum incision device of FIG. 1A. In the embodiment, the fixing part 160 includes an ear-opening element 162 and a holding element 164. The ear-opening element 162 has a perforation 162a, and the holding element 164 is disposed at an end of the perforation 162a. In the embodiment, the ear-opening element 162 is detachably inserted into the ear canal 54. The holding element 164 is disposed at an end, where the ear-opening element 162 is inserted into the ear canal 54, and may movably lean against the ear canal 54. Besides, the holding element 164 has a through hole 164a to make the replaceable inserting portion 120 inserted therein.

FIGS. 5B and 5C are an embodiment of the fixing part of the eardrum incision device of FIG. 1A. FIGS. 5A, 5B and 5C are provided for reference. As described above, the ear-opening element 162 has the perforation 162a. The replaceable inserting portion 120 may penetrate the perforation 162a to be disposed through the through hole 164a of the holding element 164 to be fixed at the target position in the ear canal 54, i.e. a position suitable for image capture by the image-capturing component 130. In the embodiment, the holding element 164 may be made of flexible silicone rubber materials and may be black to absorb light lest the holding element 164 itself should reflect light onto the image-capturing component 130, lead to detected noises for the image-capturing component 130 and further reduce the accuracy of image detection.

It is worth mentioning that the inside of the ear-opening element 162 may be stuffed with gases. When the replaceable inserting portion 120 is inserted into the ear canal 54 through the ear-opening element 162, the replaceable inserting portion 120 of the eardrum incision device 100 may squeeze the ear-opening element 162 through force exerted by the operator. Therefore, gases in the ear-opening element 162 shove in a direction toward the partial ear-opening element 162 disposed in the ear canal 54, the volume of the partial ear-opening element 162 in the ear canal 54 swells, the ear-opening element 162 leans against the ear canal 54 more securely, and meanwhile, grips the replaceable inserting portion 120 penetrating the ear-opening element 162.

FIG. 6 is a schematic view of an embodiment of the eardrum incision device of FIG. 1A. In the embodiment, a sum of distances d1 and d2 in the FIG. 6 is a working distance at which the image-capturing component 130 may capture a three-dimensional colourful image of the eardrum 52 in the ear canal 54, approximately between 0 and 10 mm. When the insertion end 120a of the eardrum incision device 100 enters within a range of the distance d1, the eardrum incision device 100 may obtain information on the depth at which the insertion end 120a of the replaceable inserting portion 120 is in the ear canal 54 according to the three-dimensional image of the inside of the ear canal 54 captured by the dual lenses of the image-capturing component 130. That is, the information is about the depth at which the insertion end 120a is in the ear canal 54 and a distance between the insertion end 120a and the eardrum 52.

In the embodiment, the distance d2 in FIG. 6 is a warning distance. That is, when the insertion end 120a enters a range of the distance d2, the eardrum incision device 100 starts issuing a warning in the way of sounds or visual images to warning the insertion end 120a of getting too close to the eardrum 52 to prevent the insertion end 120a from contacting the eardrum 52. For example, the more the insertion end 120a approaches the eardrum 52, the higher the frequency at which the eardrum incision device 100 issues a warning of sounds or visual images is. This way reminds the operator that the insertion end 120a already approaches the position of the eardrum 52 and that modifications have to be made to the depth at which the insertion end 120a is in the eardrum 52. The eardrum incision device 100 may depend on the three-dimensional image displayed by the display 180 and the frequency of a warning for the appropriate position of the insertion end 120a in the ear canal 54. The insertion end 120a may be fixed at the appropriate position through the fixing part 160.

In the embodiment, the eardrum incision device 100 may obtain information on the depth at which the insertion end 120a enters the ear canal 54 through the three-dimensional image captured by the dual lenses of the image-capturing component 130 from the ear canal 54. Therefore, the insertion end 120a may be fixed at the appropriate position in the ear canal 54 according to the actual operation demand of the eardrum incision device 100 and be prevented from contacting the eardrum 52 to damage the eardrum 52. In other words, the insertion end 120a may modify and enhance the depth at which the insertion end 120a enters the ear canal 54 and the position of the insertion end 120a in the ear canal 54 with the aid of the image-capturing component 130. Afterwards, the insertion end 120a is further fixed at the position through the fixing part 160. After the insertion end 120a is fixed at the appropriate position, the switch button 152 on a handle 150 of the eardrum incision device 100 may be pressed to drive the replaceable incision component 140 in the mounting hole 124 in the replaceable inserting portion 120 and make the replaceable inserting portion 120 pierce out through the insertion end 120a to incise the eardrum 52.

In view of the foregoing, the eardrum incision device of embodiments of the invention may capture the three-dimensional image of the inside of the ear canal through the dual lenses of the image-capturing component and obtain information on the depth at which the insertion end of the replaceable inserting portion of the eardrum incision device is in the ear canal according to the three-dimensional image. Therefore, the depth at which the insertion end of the replaceable inserting portion of the eardrum incision device is in the ear canal may be under control more accurately. The insertion end may modify and enhance the position where the insertion end is disposed according to information on the depth obtained by the three-dimensional image to prevent the insertion end from contacting the eardrum. Besides, the replaceable inserting portion and the insertion end thereof may be fixed at the position through the fixing part. Furthermore, the replaceable incision component may also incise the eardrum more accurately with the aid of the image-capturing component during the process of incision. Especially, the operator may effectively control the depth at which the eardrum is incised.

In the embodiments of the invention, a diameter of the image-capturing component is, for example, less than or equal to 4 mm. Consequently, the dual lenses of the image-capturing component may be disposed on the insertion end at the same time, and the insertion end may have the mounting hole configured to accommodate the replaceable incision component and the light source configured to provide illumination and project the fixing mark. Therefore, during the process where the eardrum incision device captures images and incises the eardrum, there is no need for replacement of the members, and image capture and eardrum incision may be conducted at the same time.

In the embodiments of the invention, the replaceable inserting portion of the eardrum incision device and the interior members may be made of disposable materials. This way facilitates the replacement of the members of the eardrum incision device and prevents the problem of infection of viruses for the user due to repeated use of the members. The user may have improvements in hygiene and convenience.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. An eardrum incision device, comprising:
a main body;
a replaceable inserting portion, detachably connected to the main body, wherein the replaceable inserting portion has an insertion end located at an end far away from the main body and is adapted to be inserted into an ear canal of a user, the replaceable inserting portion comprising:
an image-capturing component, disposed at the insertion end;
a mounting hole extending in a direction from the insertion end toward the main body; and
a replaceable incision component, removably disposed in the mounting hole and adapted to pierce out of the mounting hole in a direction toward the insertion end, so as to incise an eardrum of the user; and
a display, disposed on the main body and electrically connected to the image-capturing component to display an image captured by the image-capturing component.

2. The eardrum incision device according to claim 1, wherein the image-capturing component comprises a dual lens camera module.

3. The eardrum incision device according to claim 1, further comprising a fixing ring, disposed between the replaceable inserting portion and the main body, wherein the replaceable inserting portion is detachably fixed on the main body through the fixing ring.

4. The eardrum incision device according to claim 1, further comprising a fixing part, detachably put on the replaceable inserting portion, wherein the fixing part comprises:
an ear-opening element, detachably inserted into the ear canal; and
a holding element, disposed at an end of the ear-opening element which is inserted into the ear canal, and movably leaning against the ear canal, wherein the holding element has a through hole, and the replaceable inserting portion is disposed through the through hole to be fixed in the ear canal.

5. The eardrum incision device according to claim 1, further comprising a light source, disposed at the insertion end, wherein when the insertion end is inserted into the ear canal, the light source is adapted to provide illumination.

6. The eardrum incision device according to claim 1, further comprising a driving element, disposed in the mounting hole and coupled to the replaceable incision component, wherein the driving element is adapted to drive the replaceable incision component to pierce out of the mounting hole toward the insertion end.

7. The eardrum incision device according to claim 1, further comprising a suction element, disposed in the replaceable incision component.

8. The eardrum incision device according to claim 7, further comprising at least an ear tube, disposed at an end of the suction element.

9. The eardrum incision device according to claim 1, further comprising a gripping portion and a switch button, wherein the gripping portion is connected to the main body, and the switch button is disposed on the gripping portion.

10. The eardrum incision device according to claim 1, wherein the replaceable inserting portion is made of a disposable material.
